(19) United States
(12) Patent Application Publication
(10) Pub. No.: US 2010/0227868 A1
(43) Pub. Date: Sep. 9, 2010

(54) TREATMENT METHODS WITH BRIMONIDINE

(76) Inventor: Brent A. Johnson, Ladera Ranch, CA (US)

Correspondence Address: ALLERGAN, INC., 2525 DUPONT DRIVE, T2-7H, IRVINE, CA, 92612-1599, US

(21) Appl. No.: 12/677,880

(22) PCT Filed: Sep. 19, 2008
(86) PCT No.: PCT/US08/76994

§ 371 Date: Mar. 12, 2010

Related U.S. Application Data
(60) Provisional application No. 60/973,804, filed on Sep. 20, 2007.

(51) Int. Cl. A61K 31/498; A61P 27/02

(52) U.S. Cl. 514/249

(57) ABSTRACT

Disclosed herein are therapeutic methods related to brimonidine.

### CROSS-REFERENCE

This application claims the benefit of U.S. Provisional Application Ser. No. 60/973,804, filed Sep. 20, 2007, which is hereby incorporated by reference in its entirety.

### DETAILED DESCRIPTION OF THE INVENTION

Disclosed herein is a method of treating loss of corneal sensitivity comprising topically administering to a mammal in need thereof a composition comprising a therapeutically effective amount of brimonidine.

In one embodiment, the compositions disclosed herein are administered to an eye of a mammal in need thereof to treat loss of corneal sensitivity after surgery affecting the cornea.

In another embodiment, the compositions disclosed herein are administered to an eye of a mammal in need thereof to improve recovery of corneal sensitivity after surgery affecting the cornea.

In another embodiment, the compositions disclosed herein are administered to an eye of a mammal in need thereof to treat post herpetic loss of corneal sensitivity.

Unless otherwise indicated, the term “brimonidine” refers to the brimonidine free base, as well as any salt form.

Topical ophthalmic brimonidine compositions are currently available, and may be used to practice this method. For example, a 0.2% (w/v) topical ophthalmic brimonidine tartrate solution commercially available as Alphagan® may be administered to the eye of a person in need thereof 1-4 times a day. Other commercial compositions that may also be used are Alphagan P®, which is a 0.15% (w/v) topical ophthalmic brimonidine tartrate solution, or Alphagan Z®, which is a 0.1% (w/v) topical ophthalmic brimonidine tartrate solution.

Since brimonidine has to penetrate fewer barriers to treat the cornea as compared to reduction of intraocular pressure, a lower concentration of brimonidine may be effective. For example, concentrations from 0.0001% to 0.05% (w/v) may be effective. This may also be useful in avoiding reduction of intraocular pressure, if that is desired. It may also be effective in reducing or avoiding adverse events. Methods of preparing a lower concentration composition are well known in the art. For example, the composition of one of the commercial products could be used, except that the concentration of brimonidine tartrate would be reduced.

The treatment generally comprises administering 10-50 μL drops of the compositions disclosed herein topically to the eye or eyes of the mammal or human from 1-4 times a day.

In one embodiment, the composition is administered twice a day.

In another embodiment, the composition is administered once a day.

Loss of corneal sensitivity may be related to a number of factors. For example, loss of corneal sensitivity is often caused by surgery affecting the cornea or by viral infection.

Examples of surgery that can cause loss of corneal sensitivity include keratorefractive surgery or penetrating keratoplasty, such as the following procedures:
radial keratotomy,
photorefractive keratotomy,
laser-assisted in situ keratomileusis (LASIK),
laser assisted sub-epithelial keratomileusis (LASEK),
SB-LASIK,
EPI-LASIK,
and the like.

Examples of viral infections that can cause loss of corneal sensitivity include:
HSV-1,
HSV-2,
VZV,
and the like

For the purposes of this disclosure, “treat,” “treating,” or “treatment” refer to the use of a compound, composition, therapeutically active agent, or drug in the diagnosis, cure, mitigation, treatment, prevention of disease or other undesirable condition, or to affect the structure or any function of the body of man or other animals.

What is claimed is:

1. A method of treating loss of corneal sensitivity comprising administering a composition comprising a therapeutically effective amount of brimonidine to a person in need thereof.

2. The method of claim 1 wherein the loss of corneal sensitivity is related to surgery affecting the cornea or viral infection.

3. The method of claim 2 wherein the loss of corneal sensitivity is associated with keratorefractive surgery or penetrating keratoplasty.

4. The method of claim 3 wherein the loss of corneal sensitivity is caused by the person having radial keratotomy.

5. The method of claim 3 wherein the loss of corneal sensitivity is caused by photorefractive keratotomy.

6. The method of claim 3 wherein the loss of corneal sensitivity is caused by laser-assisted in situ keratomileusis.

7. The method of claim 3 wherein the loss of corneal sensitivity is caused by laser assisted sub-epithelial keratomileusis.

8. The method of claim 3 wherein the loss of corneal sensitivity is caused by SB-LASIK.

9. The method of claim 3 wherein the loss of corneal sensitivity is caused by EPI-LASIK.

10. The method of claim 2 wherein the loss of corneal sensitivity is caused by viral infection.

11. The method of claim 10 wherein the viral infection is HSV-1.

12. The method of claim 10 wherein the viral infection is HSV-2.

13. The method of claim 10 wherein the viral infection is VZV.

14. The method of claim 1, wherein the composition contains from 0.0001% to 0.05% (w/v) brimonidine tartrate.

15. The method of claim 1, wherein the composition contains 0.2% (w/v) brimonidine tartrate.

16. The method of claim 1, wherein the composition contains 0.15% (w/v) brimonidine tartrate.

17. The method of claim 1, wherein the composition contains 0.1% (w/v) brimonidine tartrate.
